(19) United States
(12) Patent Application Publication
(10) Pub. No.: US 2007/0292463 A1
(43) Pub. Date: Dec. 20, 2007

(54) COMPOSITIONS AND METHODS COMPRISING ENERGIZABLE PARTICLES FOR HEALTH USE

(76) Inventor: Donald Spector, New York, NY (US)

Correspondence Address: DIEHL SERVILLA LLC, 77 BRANT AVE, SUITE 210, CLARK, NJ, 07066, US

(21) Appl. No.: 11/846,111

(22) Filed: Aug. 28, 2007

Related U.S. Application Data
(63) Continuation-in-part of application No. 11/318,191, filed on Dec. 22, 2005.
(60) Provisional application No. 60/825,920, filed on Sep. 17, 2006.

(51) Int. Cl. A61K 33/24; A61K 33/26; A61K 35/00; A61K 8/19; A61K 9/127; A61K 9/14; A61K 9/50; A61N 2/00; A61Q 1/02; A61Q 17/04; A61Q 19/00

(52) U.S. Cl. 424/401; 424/450; 424/490; 424/617; 424/62; 424/63; 424/646; 424/648; 600/9

(57) ABSTRACT

The present invention provides a composition comprising a topical composition base and a plurality of particles selected from the group consisting of magnetic particles, particles comprising microcircuit chips, and combinations thereof. The composition can further comprise one or more active agents. The particles and/or active agent(s) can optionally be encapsulated and/or coated. Methods for making and using the composition are also provided.

### CROSS-REFERENCE TO RELATED APPLICATIONS

This application claims the benefit of priority under 35 U.S.C. § 119(e) to U.S. Provisional Application Ser. No. 60/825,920, filed Sep. 17, 2006, and is a continuation-in-part under 35 U.S.C. § 120 of U.S. application Ser. No. 11/318,191, filed Dec. 22, 2005, all of which are hereby incorporated by reference herein.

### BACKGROUND OF THE INVENTION

There has been considerable research showing that magnetic fields have a positive influence on health. Many of these studies have now been reported in legitimate medical journals and are used in treatments for arthritis, wellness and other specific ailments. For example, Eccles concluded that “weight of evidence from published, well-conducted controlled trials suggests that static magnetic fields are able to induce analgesia.” J. Altern. Complement. Med., 11(3):495-509 (2005). Similarly, Weintraub et al. reported that “constant wearing of multipolar, static magnetic (450G) shoe insoles helps to alleviate pain associated with symptomatic diabetic peripheral neuropathy.” Arch. Phys. Med. Rehabil. 84(5):736-46 (2003).

The market for bracelets and other jewelry has soared as magnetic theory for healing and health has become more widely accepted. However, these magnets are placed over a very small area of the body and they rely on force fields to carry them, which are non-specific in design.

There are continuing improvements in microtechnology, particularly in micro and nano circuits. In October 2004, at the CEATEC Japan, ultra-small chips measuring 0.4 by 0.2 mm, now referred to as “0402” chips were exhibited. These ultra-small chips make microcircuitry available to applications not possible previously.

Accordingly, new and improved compositions and method of using magnetic fields and electromagnetic radiation are needed.

### SUMMARY OF THE INVENTION

In one aspect, the present invention provides a topical composition comprising a topical composition base and a plurality of particles selected from the group consisting of magnetic particles, particles comprising microcircuit chips, and combinations thereof. The topical composition may also comprise one or more active agents selected from the group consisting of vitamins, antimicrobial agents, anti-inflammatory agents, soothing agents, skin lightening agents, bronzers, anti-wrinkle agents, sunscreen agents, anti-itching agents, antioxidants, fragrances, conditioners, heating agents, cooling agents, and any combination thereof. The magnetic particles and/or active agent(s) can be encapsulated, coated and/or packaged in controlled-release formulations.

In another aspect, the present invention provides for a method of treating a mammal with a condition treatable by an application of a magnetic field or electromagnetic radiation, such as pain, comprising applying to the desired area of said mammal a topical composition comprising a topical composition base and a plurality of magnetic particles. Preferably, the mammal is a human.

In yet another aspect, the present invention provides for a method of making a topical composition comprising mixing a topical composition base with a plurality of particles and, optionally, one or more active agents under conditions sufficient to form the topical composition. The magnetic particles can be magnetized prior to, during or after mixing with the base. The microcircuit particles may be energized by application of an electromagnetic radiation having a suitable frequency. For particular applications, the energizing application may be continuous or pulsed.

### BRIEF DESCRIPTION OF THE DRAWINGS

FIG. 1 illustrates a topical cream composition comprising encapsulated particles selected from the group consisting of magnetic particles, particles comprising microcircuit chips, and combinations thereof and a plurality of encapsulated active ingredients.

FIG. 2 illustrates a bandage or transdermal patch or the like with compositions in accordance with various aspects of the present invention applied to thereto.

### DETAILED DESCRIPTION OF THE INVENTION

As used in this disclosure the term electromagnetic radiation includes all wavelengths and harmonics of self-propagating waves known as the “electromagnetic spectrum” consistent with the limitations of antenna and circuitry of the present invention. The energizing application may be continuous or pulsed in a preselected frequency.

As used in this disclosure, the term “encapsulated”, “encapsulant”, “coating” or “coated” are used interchangeably, and refers to all techniques used for encapsulating or coating a particle or article. A non-exclusive list of encapsulation techniques includes microspheres, microparticles, microbubbles, liposomes and materials capable of being spray dried.

The present invention solves the shortcomings of magnetic jewelry by incorporating small particles selected from the group consisting of magnetic particles, particles comprising microcircuit chips, and combinations thereof into articles, including, but not limited to, cosmetic or pharmaceutical topical compositions. Thus, the magnetic fields and electromagnetic radiation that emanate from the particles in the topical composition of the invention may be applied to a more specific surface area thus better serving the needs of the user than, for example, wearing an anklet or a bracelet. When applied as a topical composition, the magnetic fields or generated electromagnetic radiation will be generated directly on the surface of the skin, therefore improving the application of the field to a subject, which is important because the magnetic fields diminish rapidly with distance (according to an inverse square of the distance) and the electromagnetic radiation that can be generated by a microcircuit may have low intensity or power.

One aspect of the present invention provides a composition comprising a topical composition base and a plurality of particles selected from the group consisting of magnetic particles, particles comprising microcircuit chips, and combinations thereof. Non-limiting examples of materials suitable for the magnetic particles include iron, Fe₂O₃, other ferromagnetic elements and compositions, nickel, cobalt, neodymium, samarium or any mixture thereof. Non-limiting examples of microcircuit chips include, but are not limited to, a chip condenser, a chip bead, a chip varistor, ultra-small, next generation chips such as 0402 chips available from TDK, Japan, and the like, or super-magnetorestrictive materials formed into coils and combinations of microchips and coils formed from super-magnetorestrictive materials.

The 0402 chips measure about 0.4 mm by about 0.2 mm and in one embodiment may possibly be energized with electromagnetic radiative energy received through a high frequency or ultrahigh frequency antenna formed from a screen printable conductive ink containing silver. One suitable silver containing ink is available from. DuPont Microcircuit Materials, Wilmington, Del. under stock number 5033. The use of this or similar conductive screen printable inks to form an antenna may permit sufficient energy transfer to energize the encapsulated microcircuits.

Super-magnetorestrictive materials may be formed into coils wrapped around a rod. When a current is passed through the rod, the dimensions of the rod may change and can cause a dimension change that not only causes vibration, but also raises frequency and generates ultrasonic waves when a current is passed through the rod. Suitable super-magnetorestrictive materials include those displayed by TDK at the CEATEC Japan 2004 exposition. According to the supplier, TDK, A material is designated as super-magnetorestitive if it is capable of causing a change in dimension more than 50 times greater than that produced in a conventional magnetostrictive material.

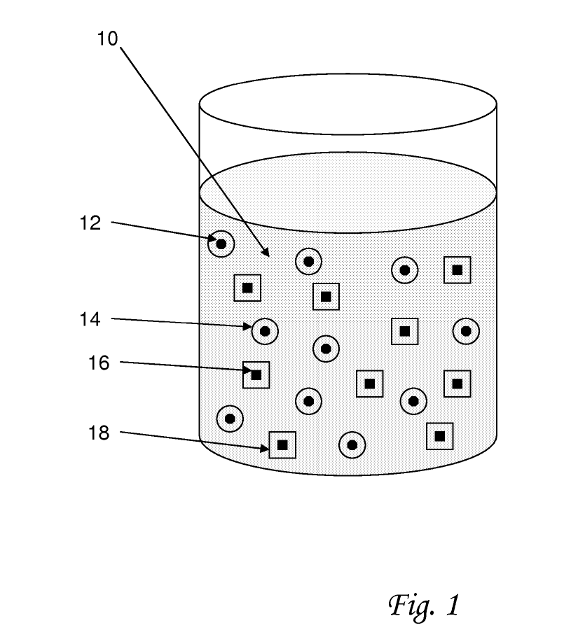

FIG. 1 illustrates one aspect of the present invention. A cream base 10 is provided. A plurality of particles selected from the group consisting of magnetic particles, particles comprising microcircuit chips, and combinations thereof 12 are mixed into the cream base. The plurality of particles can be encapsulated with an encapsulant 14. One or more active agents 16 can also be mixed into the cream base 10. The one or more active agents 16 can also be encapsulated with an encapsulant 18. Further, the cream base 10 can be a liquid. The particles selected from the group consisting of magnetic particles, particles comprising microcircuit chips, and combinations thereof preferably comprise from about 0.001% to about 10% w/w of the topical composition. In one embodiment, the particles comprise between about 0.001 to about 5% w/w of the topical composition.

The topical composition of various aspects of the invention can be in a solid or liquid form. Suitable solid topical compositions include, for example, powders, sticks or bars similar to deodorant sticks. Suitable liquid topical compositions include, for example, gels, dispersions, microemulsions, emulsions, suspensions, foams, mousses, pastes, creams, balms, ointments, body washes, rinses, lotions, oils and sprays.

A person of ordinary skill in the art will appreciate that the compositions of various aspects of the present invention may be formulated with emollients, solvents, thickeners, humectants and/or powders. Suitable emollients include, but are not limited to, stearyl alcohol, mink oil, cetyl alcohol, oleyl alcohol, isopropyl laurate, polyethylene glycol, olive oil, petroleum jelly, palmitic acid, oleic acid, and myristyl myristate.

Suitable solvents include, but are not limited to, ethyl alcohol, isopropyl alcohol, acetone, diethylene glycol, ethylene glycol, dimethyl sulfoxide, and dimethyl formamide.

Suitable humectants include, but are not limited to, acetyl arginine, algae extract, aloe barbadensis leaf extract, 2,3-butanediol, chitosan lauroyl glycinate, diglycereth-7 malate, diglycerin, diglycol guanidine succinate, erythritol, fructose, glucose, glycerin, honey, hydrolyzed wheat protein/polyethylene glycol-20 acetate copolymer, hydroxypropyltrimonium hyaluronate, inositol, lactitol, maltitol, maltose, mannitol, mannose, methoxy polyethylene glycol, myristamidobutyl guanidine acetate, polyglyceryl sorbitol, potassium pyrollidone carboxylic acid (PCA), propylene glycol, sodium pyrollidone carboxylic acid (PCA), sorbitol, sucrose, and urea. Other humectants may be used for yet additional embodiments of this invention, as will be appreciated by those skilled in the art.

Suitable thickeners include, but are not limited to, polysaccharides, in particular xantham gum, guar-guar, agar-agar, alginates, carboxymethylcellulose, relatively high molecular weight polyethylene glycol mono- and diesters of fatty acids, polyacrylates, polyvinyl alcohol and polyvinylpyrrolidone, surfactants such as, for example, ethoxylated fatty acid glycerides, esters of fatty acids with polyols such as, for example, pentaerythritol or trimethylpropane, fatty alcohol ethoxylates or alkyl oligoglucosides, and electrolytes, such as sodium chloride and ammonium chloride.

The compositions of various embodiments of the present invention may also include components that enhance the penetration of the magnetic particles into the skin and/or to the site of action. Suitable examples of penetration enhancers include, but are not limited to, urea, propan-2-ol, polyoxyethylene ethers, terpenes, cis-fatty acids (oleic acid, palmitoleic acid), acetone, laurocapram dimethyl sulfoxide, 2-pyrrolidone, oleyl alcohol, glyceryl-3-stearate, cholesterol, myristic acid isopropyl ester, and propylene glycol.

The methods of formulation of different topical compositions are well known to a skilled artisan. For example, the base of the topical composition described in some aspects of the instant disclosure may be formulated such as a cream, an ointment, an aerosol formulation, a non-aerosol spray, a gel, a foam, a solution, a suspension, a dispersion, an emulsion, a microemulsion, a paste, a powder, a solid stick (e.g., wax- or petroleum-based sticks), a wipe, an oil, a lotion, and the like. For administration, the cosmetic and dermatological preparations of the invention may be applied to the skin in adequate quantity in the manner conventional for cosmetics. A person skilled in the art can select the appropriate presentation form, and also the method for preparing it, on the basis of his general knowledge, taking into account on the one hand the nature of the constituents used, and on the other hand the intended use of the composition.

In some embodiments, in addition to the plurality of particles selected from the group consisting of magnetic particles, particles comprising microcircuit chips, and combinations thereof, one or more active agents are included the topical composition. The particles and the active agent(s) may perform the same or different functions. For example, vitamins, antimicrobial agents, anti-inflammatory agents, soothing agents, non-steroidal cosmetic soothing agents, skin lightening agents, anti-wrinkle agents, sunscreen agents, anti-itching agents, antioxidants, fragrances, conditioners, heating agents, cooling agents, bronzers, and other agents or any combination thereof. The active agent(s) preferably comprise from about 0.001% to about 10% w/w of the topical composition.

Non-limiting examples of vitamins useful herein include, but are not limited to, vitamin A, vitamin B₁-B₁₂, biotin, vitamin C, pantothenic acid, vitamin K, vitamin D, vitamin E and mixtures thereof.

Non-limiting examples of antimicrobial agents useful herein include, but are not limited to, beta-lactam drugs, quinolone drugs, ciprofloxacin, norfloxacin, tetracycline, erythromycin, amikacin, 2,4,4′-trichloro-2′-hydroxy diphenyl ether, 3,4,4′-trichlorobanilide, phenoxyethanol, phenoxy propanol, phenoxyisopropanol, doxycycline, capreomycin, chlorhexidine, chlortetracycline, oxytetracycline, clindamycin, ethambutol, hexamidine isethionate, metronidazole, pentamidine, gentamicin, kanamycin, lineomycin, methacycline, methenamine, minocycline, neomycin, netilmicin, paromomycin, streptomycin, tobramycin, miconazole, tetracycline hydrochloride, erythromycin, zinc erythromycin, erythromycin estolate, erythromycin stearate, amikacin sulfate, doxycycline hydrochloride, capreomycin sulfate, chlorhexidine gluconate, chlorhexidine hydrochloride, chlortetracycline hydrochloride, oxytetracycline hydrochloride, clindamycin hydrochloride, ethambutol hydrochloride, metronidazole hydrochloride, pentamidine hydrochloride, gentamicin sulfate, kanamycin sulfate, lineomycin hydrochloride, methacycline hydrochloride, methenamine hippurate, methenamine mandelate, minocycline hydrochloride, neomycin sulfate, netilmicin sulfate, paromomycin sulfate, streptomycin sulfate, tobramycin sulfate, miconazole hydrochloride, amanfadine hydrochloride, amanfadine sulfate, octopirox, parachlorometa xylenol, nystatin, tolnaftate, zinc pyrithione; clotrimazole; alantolactone; isoalantolactone; alkanet extract (alaninin); anise; arnica extract (helenalin acetate and 11,13 dihydrohelenalin); Aspidium extract (phloro, lucinol containing extract); barberry extract (berberine chloride); bay sweet extract; bayberry bark extract (myricitrin); benzalkonium chloride; benzethonium chloride; benzoic acid and its salts; benzoin; benzyl alcohol; blessed thistle; bletilla tuber; bloodroot; bois de rose oil; burdock; butyl paraben; cade oil; CAE (available from Ajinomoto, located in Teaneck, N.J.); cajeput oil; Cangzhu; capsicum frutescens extract; caraway oil; cascarilla bark (sold under the tradename ESSENTIAL OIL); cedarleaf oil; chamomille; chaparral; chlorhexidine gluconate; chlorophenesin; chlorxylenol; cinnamon oil; citronella oil; clove oil; Crinipan AD (available from Climbazole); 2,3-dihydro-famesol; dehydroacetic acid and its salts; dill seed oil; DOWICIL 200 (available from Dow Chemical, located in Midland, Mich.); echinacea; elenolic acid; epimedium; ethyl paraben; Fo-Ti; galbanum; garden bumet; GERMALL 115 and GERMALL II (available from ISP-Sutton Labs, located in Wayne, N.J.); German chamomile oil; giant knotweed; GLYDANT (available from Lonza, located in Fairlawn, N.J.); GLYDANT PLUS (available from Lonza); grapefruit seed oil; 1,6 hexanediol; hexamidine diisethionate; hinokitiol; honey; honeysuckle flower; hops; immortelle; iodopropynl butyl carbamide (available from Lonza); isobutyl paraben; isopropyl paraben; JM ACTICARE (available from Microbial Systems International, located in Nottingham, NG); juniper berries; KATHON CG (available from Rohm and Haas, located in Philadelphia, Pa.); kojic acid; labdanum; lavender; lemon balm oil; lemon grass; methyl paraben; mint; mume; mustard; myrrh; neem seed oil; ortho phenyl phenol; olive leaf extract (available from Bio Botanica); parsley; patchouly oil; peony root; 1,2 pentandiol; PHENONIP (available from Nipa Labs, located in Wilmington, Del.); phenoxyethanol; phytosphingosine; pine needle oil; PLANSERVATIVE (available from Campo Research); propyl paraben; purslane; quillaira; rhubarb; rose geranium oil; rosemary; sage; salicylic acid; sassafras; savory; sichuan lovage; sodium meta bisulfite; sodium sulfite; SOPHOLIANCE (available from Soliance, located in Compiegne, France); sorbic acid and its salts; sphingosine; stevia; storax; sucrose esters; tarmic acid; tea; tea tree oil (cajeput oil); thyme; triclosan; triclocarban; tropolone; turpentine; umbelliferone (antifungal); yucca; and mixtures thereof.

Non-limiting examples of anti-inflammatory agents useful herein include, but are not limited to, hydrocortisone, non-steroidal anti-inflammatory agents such as oxicans, salicylates, acetic acid derivatives, fenamates, propionic acid derivatives, pyrazoles, substituted phenyl compounds, 2-naphthyl containing compounds, and natural anti-inflammatory agents such as aloe vera. Examples of anti-inflammatory agents are described in U.S. Pat. No. 5,487,884, the entire contents of which is incorporated herein by reference.

Non-limiting examples of useful cosmetic soothing agents useful herein include, but are not limited to, acetyl salicylic acid, ibuprofen, naproxen, benoxaprofen, flurbiprofen, fenoprofen, fenbufen, ketoprofen, indoprofen, pirprofen, carprofen, oxaprozin, pranoprofen, miroprofen, tioxaprofen, suprofen, alminoprofen, tiaprofenic acid, fluprofen, bucloxic acid, absinthium, acacia, aescin, alder buckthorn extract, allantoin, aloe, APT (available from Centerchem), amica, astragalus, astragalus root extract, azulene, Baicalin SR 15 (available from Barnet Products Dist.), baikal skullcap, baizhu, balsam canada, bee pollen, BIOPHYTEX (available from Laboratories Serobiologiques), bisabolol, black cohosh, black cohosh extract blue cohosh, blue cohosh extract, boneset, borage, borage oil, bradykinin antagonists, bromelain, calendula, calendula extract, Canadian Willowbark Extract (available from Fytokem), candelilla wax, Cangzhu, canola phytosterols, capsicum, carboxypeptidase, celery seed, celery stem extract, CENTAURIUM (available from Sederma), centaury extract, chamazulene, chamomile, chamomile extract, chaparral, chaste tree, chaste tree extract, chickweed, chicory root, chicory root extract, chirata, chishao, collodial oatmeal, comfrey, comfrey extract, CROMOIST CM GLUCAN (available from Croda), darutoside, dehurian angelica, devil's claw, divalent metals (such as, magnesium, strontium, and manganese), doggrass, dogwood, Eashave (available from Pentapharm), eleuthero, ELHIBIN (available from Pentapharm), ENTELINE 2 (available from Secma), ephedra, epimedium, esculoside; ethacrynic acid, evening primrose, eyebright, Extract LE-100 (available from Sino Lion), Fangfeng, feverfew, ficin, forsythia fruit, Fytosterol 85 (available from Fytokem), ganoderma, gaoben, Gatuline A (available from Gattefosse), gentian, germanium extract, gingko bilboa extract, ginkgo, ginseng extract, goldenseal, gorgonian extract, gotu kola, grape fruit extract, guaiac wood oil, guggal extract, helenalin esters, henna, honeysuckle flower, horehound extract, horsechestnut, horsetail, huzhang, hypericum, ichthyol, immortelle, ipecac, job's tears, jujube, kola extract, LANACHRYS 28 (available from Lana Tech), lemon oil, lianqiao, licorice root, ligusticum, ligustrum, lovage root, luffa, mace, magnolia flower, manjistha extract, margaspidin, matricin, melatonin, MICROAT IRC (available from Nurture), mints, mistletoe, Modulene (available from Seporga), mono or diglucosides of glabridin, mono or diglucosides of gentisin, MTA (5′-deoxy-5′-methythioadenosine), mung bean extract, musk, N-methyl arginine, oat beta glucan, oat extract, orange, panthenol, papain, phenoxyacetic acid, peony bark, peony root, Phytoplenolin (available from Bio Botanica), phytosphingosine, Preregen (available from Pentapharm), purslane, QUENCH T (available from Centerchem), quillaia, red sage, rehmannia, rhubarb, rosemary, rosmarinic acid, royal jelly, rue, rutin, sandlewood, sanqi, sarsaparilla, saw palmetto, SENSILINE (available from Silab), SIEGESBECKIA (available from Sederma), stearyl glycyrrhetinate, Stimutex (available from Pentapharm), storax, strontium nitrate, sweet birch oil, sweet woodruff, tagetes, tea extract, thyme extract, tienchi ginseng, tocopherol, tocopheryl acetate, triclosan, turmeric, urimei, ursolic acid, white pine bark, witch hazel xinyi, yarrow, yeast extract, yucca, and mixtures thereof.

Non-limiting examples of skin lightening agents useful herein include, but are not limited to, adapalene, aloe extract, alpha-glycaryl-L-ascorbic acid, aminotyroxine, ammonium lactate, anethole derivatives, apple extract, arbutin, areca catechu L. extract, ascorbic acid, ascorbyl palmitate, azelaic acid, bamboo extract, bearberry extract, bletilla tuber, bupleurum falcatum extract, bumet extract, Bumet Power (available from Barnet Products), butyl hydroxy anisole, butyl hydroxy toluene, butyl resoreinol, Chuanxiong, cola decaballo extract, Dang-Gui, deoxyarbutin, 1,3 diphenyl propane derivatives, 2,5 dihydroxybenzoic acid and its derivatives, 2-(4-acetoxyphenyl)-1,3 dithane, 2-(4-hydroxyphenyl)-1,3 dithane, ellagic acid, escinol, estragole derivatives, esculoside, esculetin, FADEOUT (available from Pentapharm), Fangfeng, fennel extract, gallic acid and its derivatives, ganodenna extract, gaoben, GATULINE WHITENING (available from Gattlefosse), genistic acid and its derivatives, gentisyl alcohol, glabridin and its derivatives, gluco pyranosyl-1-ascorbate, gluconic acid, glucosamine, glycolic acid, glycyrrhizinic acid, green tea extract, 4-Hydroxy-5-methyl-3[2H]-furanone, hydroquinine, 4 hydroxyanisole and its derivatives, 4-hydroxy benzoic acid derivatives, hydroxycaprylic acid, hyptis extract, inositol ascorbate, kojic acid, kojic dipalnitate, lactic acid, lemon extract, licorice extract, Licorice P-TH (available from Barnet Products), linoleic acid, magnesium ascorbyl phosphate, Melfade (available from Pentapharm), MELAWHITE (available from Pentapharm), Melanostatine DM (available from Laboratories Seporga), morus alba extract, mulberry root extract, niacinamide, 5-octanoyl salicylic acid, parsley extract, phellinus linteus extract, pinon blanco extract, pinon negro extract, piri-piri extract, pyrogallol derivatives, retinoic acid, retinol, retinyl esters (acetate, propionate, palmitate, linoleate), 2,4 resorcinol derivatives, 3,5 resorcinol derivatives, rose fruit extract, rucinol, salicylic acid, Song-Yi extract, Sophora Powder (available from Barnet Products), 4-thioresorein, 3,4,5 trihydroxybenzyl derivatives, tranexamic acid, tyrostat (Rumex Extract available from Fytokem), Tyroslat 10,11 (available from Fytokem), vanilla derivatives, vitamin D₃ and its analogs, and mixtures thereof.

Non-limiting examples of suitable sunscreening agents useful herein include, but are not limited to, p-aminobenzoic acid, its salts and derivatives, anthranilates, salicylates, cinnamic acid derivatives, dihydroxycinnamic acid derivatives, trihydroxycinnamic acid derivatives, hydrocarbons, dibenzalacetone and benzalacetophenone, naphthosulfonates, dihydroxy-naphthoic acid and its salts, o- and p-hydroxy-biphenyldisulfonates, coumarin derivatives, diazoles quinine salts, quinoline derivatives, hydroxy or methoxy substituted benzophenones, uric and vilouric acids, tannic acid and its derivatives, hydroquinone, benzophenones, 2-ethylhexyl p-methoxycinnamate, 2-ethylhexyl N,N-dimethyl-p-aminobenzoate, p-aminobenzoic acid, 2-phenylbenzimidazole-5-sulfonic acid, octocrylene, oxybenzone, homomenthyl salicylate, octyl salicylate, 4,4′-methoxy-t-butyldibenzoylmethane, 4-isopropyl dibenzoylmethane, 3-benzylidene camphor, 3-(4-methylbenzylidene) camphor, titanium dioxide, zinc oxide, silica, iron oxide, and mixtures thereof. Still other useful sunscreens are those disclosed in U.S. Pat. Nos. 4,937,370 and 4,999,186, each of which is incorporated by reference herein in its entirety. Still other useful sunscreens include, but are not limited to, aminobenzoic acid (PABA), benzylidene camphor, butyl methoxy dibenzoyl methane, diethanolamine p-methoxycinnamate, dioxybenzone, ethyl dihydroxypropyl (PABA), glyceryl aminobenzoate, homomenthyl salicylate, isopropyl dibenzoyl methane, lawsone and dihydroxyacetone, menthyl anthranilate, methyl anthranilate, methyl benzylidene camphor, octocrylene, octyl dimethyl PABA, octyl methoxycinnamate, oxybenzone, 2-phenylbenzimidazole-5-sulfonic acid, red petrolatum, sulisobenzone, titanium dioxide, triethanolamine salicylate, zinc oxide, and mixtures thereof Especially preferred examples of these sunscreens include those selected from the group consisting of 4-N,N-(2-ethylhexyl)methylaminobenz-oic acid ester of 2,4-dihydroxybenzophenone, 4-N,N-(2-ethylhexyl)methylami-nobenzoic acid ester with 4-hydroxydibenzoylmethane, 4-N,N-(2-ethylhexyl)-methylaminobenzoic acid ester of 2-hydroxy-4-(2-hydroxyethoxy)benzophenone, 4-N,N-(2-ethylhexyl)-methylaminobenzoic acid ester of 4-(2-hydroxyethoxy)dibenzoylmethane, and mixtures thereof.

Non-limiting examples of anti-itch ingredients useful herein include, but are not limited to, Stimu-tex (available from Pentapharm); Takanal (available from Ikeda-Distributer); Ichthyol (available from International Sourcing-Distributor); Oxygenated Glyceryl Triesters (available from Seporgia) and mixtures thereof.

Non-limiting example of antioxidants useful herein include, but are not limited to, vitamin E, tocopheryl acetate, betaglucan, coenzyme Q1O, butylated hydroxy toluene (BHT), butylated hydroxy anisole BHA, superoxide dismutose, propylgallate, and the like.

Non-limiting examples of skin conditioners useful herein include, but are not limited to, mineral oil, petrolatum, vegetable oils (such as soybean or maleated soybean oil), dimethicone, dimethicone copolyol, cationic monomers and polymers (such as guar hydroxypropyl trimonium chloride and distearyl dimethyl ammonium chloride) as well as combinations thereof. Illustrative moisturizers are polyols such as sorbitol, glycerin, propylene glycol, ethylene glycol, polyethylene glycol, polypropylene glycol, 1,3-butane diol, hexylene glycol, isoprene glycol, xylitol, fructose and mixtures thereof.

Suitable fragrances which can be used in the present invention comprise, for example, the high boiling components of woody/earthy bases containing exotic materials such as sandalwood oil, civet, patchouli oil, and the like. The fragrances can be of a light, floral fragrance, such as for example, high boiling components of rose extract, violet extract, and the like. The fragrances can be formulated to provide desirable fruity odors, such as for example lime, lemon, orange, and the like. The fragrance can be any material of appropriate chemical and physical properties which exudes a pleasant or otherwise desirable odor when applied to skin. Materials suitable for use in the present invention are described more fully in S. Arctander, Perfume Flavors and Chemicals, Vols. I and II, Aurthor, Montclair, N.J. and the Merck Index, 8th Edition, Merck & Co., Inc. Rahway, N.J., each of which is incorporated herein by reference in it entirety.

Suitable non-limiting examples of skin heating agents useful herein include, but are not limited to, polyvinyl amine, polyalkyleneamine or polyalkyleneimine, as described in U.S. Patent Publication No. 20040253198, which is incorporated by reference herein in its entirety.

Suitable non-limiting examples of skin cooling agents useful herein include, but are not limited to, menthol, menthol esters, methyl salicylate, camphor, benzocaine, dibucaine, dyclonine, lidocaine, pramoxine, tetracaine, ephedrine, epinephrine, phenylephrine, and their derivatives, and combinations thereof as described in the U.S. Patent Publication 20040175340, which is incorporated herein by reference in its entirety.

Non-limiting examples of bronzers useful herein include, but are not limited to, juglone, lawsone, and Caymen Sun Secrets® Skin Bronzer.

In some embodiments, the magnetic particles and/or the active agent(s) are encapsulated before inclusion in the topical composition. Suitable encapsulating compounds and techniques of encapsulation of compounds for topical compositions are well known in the art and described, for example, in U.S. Pat. Nos. 4,478,822, 4,803,195, 4,917,982, and 5,861,440, each of which is incorporated by reference herein in its entirety. Such encapsulation agents include, for example, microspheres, microparticles, microbubbles and liposomes. Encapsulation also refers to coatings, for example, spray dried materials are considered coatings for the purposes of this disclosure.

The active agent(s) may also be delivered in a controlled-release form, such as, for example, microspheres and/or nanospheres as described for example, in U.S. Pat. No. 6,589,562 and U.S. Patent Publication Nos. 20030198652 and 20030232091, each of which is incorporated herein by reference in its entirety. It is possible to design the encapsulation mode and to select encapsulants in such a way that they will react to environmental changes and release the packaged compounds. For example, if it is desirable to create a system wherein the release of a compound encapsulated in a microsphere is triggered by increased moisture, the following materials would be suitable for the microsphere: water soluble and water dispersible synthetic polymers and copolymers, starch derivatives, polysaccharides, hydrocolloids, natural gums, proteins, and mixtures thereof. If the changes in the pH are used for triggering the release of the encapsulated compounds, a skilled artisan may use pH-sensitive substrates, such as, for example, polyacrylamides, phthalate derivatives such as acid phthalates of carbohydrates, amylose acetate phthalate, cellulose acetate phthalate, other cellulose ester phthalates, cellulose ether phthalates, hydroxypropylcellulose phthalate, hydroxypropylethylcellulose phthalate, hydroxypropylmethylcellulose phthalate, methylcellulose phthalate, polyvinyl acetate phthalate, polyvinyl acetate hydrogen phthalate, sodium cellulose acetate phthalate, starch acid phthalate, styrene-maleic acid dibutyl phthalate copolymer, styrene-maleic acid polyvinylacetate phthalate copolymer, styrene and maleic acid copolymers, polyacrylic acid derivatives such as acrylic acid and acrylic ester copolymers, polymethacrylic acid and esters thereof, poly acrylic methacrylic acid copolymers, shellac, and vinyl acetate and crotonic acid copolymers.

In yet other embodiments, the magnetic particles of the composition may additionally, or alternatively, be coated with a coating that optionally comprises a tinting agent. Suitable coating materials include, for example, the same coloration and pigment materials used in the normal practice of makeup, thereby not appearing discontinuous with normal foundation makeup. Suitable tinting agents include, for example, zinc oxide, kaolin, mica, iron oxide, titanium dioxide (U.S. Pat. No. 6,022,532), anthraquinone compounds (U.S. Pat. No. 6,660,284) and octinoxate.

In various embodiments of the invention, the magnetic particles of the composition are placed on an article. Non-limiting examples of suitable articles include bandages, textiles and transdermal patches. In some embodiments, the article is placed in close proximity to the skin, allowing contact with magnetic particles of the composition. For example, an injured ankle could be treated by placing the composition on a sock, thereby keeping the composition touching the surface of the skin at the point of interest. In another example, the composition could be put on the surface of a bandage. The bandage would hold the composition against a desired location, such as a wound.

In further embodiments, the composition comprising magnetic particles, and other optional ingredients, is put into or onto a transdermal patch or bandage that creates an electromagnetic or magnetic field. Still further embodiments employ magnetic inks or magnetic toners on the patch.

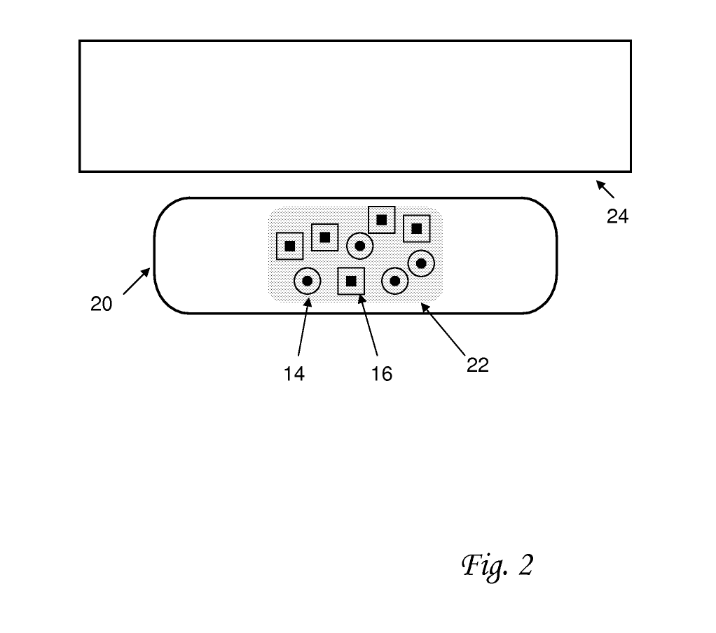

These additional embodiments are illustrated in FIG. 2. A bandage or transdermal patch or the like 20 is illustrated. The patch 20 may have an area of gauze 22 or other absorbent material. Any of the compositions of the present invention can be applied to the area of gauze 22. The patch 20 can be packaged in a package 24. When the patch 20 is removed from the package 24 and applied to the skin of a person, the compositions from the gauze area 22 are released into the skin. Additionally, the bandage, transdermal patch or the like 20 can be printed with a magnetic ink or magnetic toner.

The methods of mixing particles selected from the group consisting of magnetic particles, particles comprising microcircuit chips, and combinations thereof and, optionally, active agent(s) into a topical composition base are within the expertise of a person of ordinary skill in the art. In general, a topical composition base is mixed with a plurality of particles selected from the group consisting of magnetic particles, particles comprising microcircuit chips, and combinations thereof and, optionally, one or more active agents under conditions sufficient to form the topical composition. If the particles selected from the group consisting of magnetic particles, particles comprising microcircuit chips, and combinations thereof and/or active agents are encapsulated or coated, such encapsulation or coating is preferably done prior to mixing with topical composition base. In various embodiments, the particles can be magnetized in a suitable container (e.g., bottle) before, during or following mixing with the topical composition base. In embodiments of the invention where the topical composition includes particles having microcircuit chips, and combinations thereof with magnetic particles, the microcircuit chips may be activated by application of sufficient electromagnetic radiation of a preselected wavelength. For particular applications, the activation radiation may be applied continuously or in pulses of a preselected duration.

The topical composition of the present invention is useful for treatment of conditions treatable with an application of magnetic field or electromagnetic radiation. Depending on the form of the topical composition selected by the user, the user would rub or spray this composition onto a desired skin area. For example, if the user has pain associated with diabetic neuropathy, she can apply the topical composition of the present invention to her feet. In this non-limiting example, the topical composition of the present invention would be especially suitable for applications in situations where the user cannot or would not wear shoes with magnetic soles, such as, for example, in bed. In various embodiments the function or penetration of active agents may be enhanced or facilitated by the applied magnetic field or the electromagnetic radiation.

Although the invention herein has been described with reference to particular aspects and embodiments, it is to be understood that these aspects and embodiments are merely illustrative of the principles and applications of the present invention. It is therefore to be understood that numerous modifications may be made to the illustrative embodiments and that other arrangements may be devised without departing from the spirit and scope of the present invention as defined by the appended claims and their equivalents

What is claimed:

1. An article comprising a topical composition base and a plurality of particles selected from the group consisting of magnetic particles, particles comprising microcircuit chips, and combinations thereof.

2. The article of claim 1, wherein the microcircuit chips are selected from the group consisting of a chip condenser, a chip bead, super-magnetostrictive actuator and a chip varistor and the magnetic particles are selected from the group consisting of iron, Fe₂O₃, other ferromagnetic elements and compositions, nickel, cobalt, neodymium, samarium and combinations thereof.

3. The article of claim 1 wherein the microcircuit chip comprises an antenna formed from a silver ink.

4. The article of claim 1, wherein the magnetic particles and the microcircuit chips comprise from about 0.001% to about 10% w/w of said topical composition.

5. The article of claim 1, wherein the magnetic particles and the microcircuit chip are encapsulated within a coating selected from the group consisting of microspheres, microparticles, microbubbles, liposomes and spray dried materials.

6. The article of claim 1, further comprising one or more active agents selected from the group consisting of vitamins, antimicrobial agents, anti-inflammatory agents, soothing agents, skin lightening agents, bronzers, anti-wrinkle agents, sunscreen agents, anti-itching agents, antioxidants, fragrances, conditioners, heating agents, cooling agents and combination thereof.

7. The article of claim 6, wherein the active agent or agents comprises from about 0.001% to about 10% w/w of said topical composition.

8. The article of claim 6, wherein the active agent or agents is coated.

9. The article of claim 6, wherein the active agent or agents is encapsulated in a coating selected from the group consisting of microspheres, microparticles, microbubbles, liposomes and spray dried materials.

10. The article of claim 1, wherein the article is in a solid form.

11. The article of claim 10, wherein the article is in a solid form selected from the group consisting of sticks, bars and powders.

12. The article of claim 1, wherein the article is in a liquid form.

13. The article of claim 12, wherein the article is in a liquid form selected from the group consisting of gels, dispersions, emulsions, microemulsions, suspensions, foams, mousses, pastes, creams, balms, ointments, body washes, rinses, lotions, oils and sprays.

14. The article of claim 1, wherein the article is selected from the group consisting of a topical composition, bandages, adhesive patches, transdermal patches and textiles which can be held in proximity to skin.

15. A method of treating a mammal having a condition treatable by magnetic or electromagnetic radiative therapy comprising applying to a desired area of said mammal an article comprising a topical composition base and a plurality of particles selected from the group consisting of magnetic particles, particles comprising microcircuit chips, and combinations thereof; and applying sufficient electromagnetic energy to the area of the mammal having the article thereon to energize the microcircuit chips.

16. The method of claim 15, wherein the applying sufficient electromagnetic energy step is a continuous application.

17. The method of claim 15, wherein the applying sufficient electromagnetic energy step is an application of a pulse or pulses of electromagnetic energy having a preselected duration.

18. The method of claim 15, wherein the mammal is a human.

19. The method of claim 15, wherein the condition is pain.

20. The method of claim 15, wherein the particles are encapsulated in a coating selected from the group consisting of microspheres, microparticles, microbubbles, liposomes and spray dried materials.

21. The method of claim 15, wherein the composition further comprises one or more active agents selected from the group consisting of vitamins, antimicrobial agents, anti-inflammatory agents, soothing agents, skin lightening agents, bronzers, anti-wrinkle agents, sunscreen agents, anti-itching agents, antioxidants, fragrances, conditioners, heating agents, cooling agents and combination thereof.

22. The method of claim 21, wherein the active agent or agents is encapsulated.

23. The method of claim 22, wherein the active agent or agents is encapsulated in a coating selected from the group consisting of microspheres, microparticles, microbubbles, liposomes and spray dried materials.

24. The method of claim 22, wherein the article is selected from the group consisting of a topical composition, a bandage, an adhesive patch and a textile which can be held in proximity to skin.

25. A method of making an article comprising mixing a topical composition base with a plurality of particles selected from the group consisting of magnetic particles, particles comprising microcircuit chips, and combinations thereof under conditions sufficient to form said topical composition.

26. The method of claim 25, further comprising mixing the topical composition base with one or more active agents selected from the group consisting of vitamins, antimicrobial agents, anti-inflammatory agents, soothing agents, skin lightening agents, bronzers, anti-wrinkle agents, sunscreen agents, anti-itching agents, antioxidants, fragrances, conditioners, heating agents, cooling agents and combinations thereof.

27. The method of claim 25, further comprising applying the mixed topical composition base to an item which can be held in proximity to skin.

28. The method of claim 27, wherein the item is selected from the group consisting of bandages, adhesive patches, transdermal patches and textiles.

29. The method of claim 25, wherein the magnetic particles are magnetized prior to mixing with the topical composition base.

30. The method of claim 25, wherein the magnetic particles are magnetized during or after mixing with the topical composition base.

31. A composition comprising a topical composition base and a plurality of coated particles selected from the group consisting of magnetic particles, particles comprising microcircuit chips, and combinations thereof.

32. The composition of claim 31, wherein particles are coated with a coating having a coloration continuous with normal foundation makeup.

33. The topical composition of claim 31, wherein the coating comprises a tinting agent.
